(19) United States
(12) Patent Application Publication
(10) Pub. No.: US 2020/0178903 A1
(43) Pub. Date: Jun. 11, 2020

(54) PATIENT MONITORING SYSTEM AND METHOD HAVING SEVERITY PREDICTION AND VISUALIZATION FOR A MEDICAL CONDITION

(71) Applicant: General Electric Company, Schenectady, NY (US)

(72) Inventors: Rupanjali Chaudhuri, Bangalore (IN); Rohit Pardasani, Bangalore (IN); Hariharan Ravishankar, Bangalore (IN); Guy Vesto, Barrington, IL (US)

(73) Assignee: General Electric Company, Schenectady, NY (US)

(21) Appl. No.: 16/215,128

(22) Filed: Dec. 10, 2018

(51) Int. Cl. A61B 5/00; A61B 5/08; A61B 5/021; A61B 5/0245; A61B 5/1455; G06N 20/00; G16H 50/30

(52) U.S. Cl. CPC A61B 5/7264; A61B 5/0816; A61B 5/082; A61B 5/7275; A61B 5/7445; G16H 50/30; A61B 5/021; A61B 5/0245; A61B 5/14551; G06N 20/00; A61B 5/746

(57) ABSTRACT

A method of monitoring a patient with respect to a particular medical condition includes providing a machine learning model trained to assign a weight to each of a predefined set of features so as to calculate a risk severity index of a particular medical condition. A long time interval of time-synchronized parameter data is received for each of at least two physiological parameters, and the long time interval is divided into multiple segments each containing a predefined time increment of the parameter data. A set of feature values are determined for the segment based on the parameter data therein, including a feature value for each of the predefined set of features related to the particular medical condition. With the trained machine learning model, assigning a weight to each of the predefined set of features, and then a risk severity index of the particular medical condition is calculated for the long time interval based on the set of feature values.

### BACKGROUND

The present disclosure generally relates to patient monitoring systems and methods involving measurement of two or more physiological parameters and, more specifically, to methods and systems for tracking, visualizing, and predicting the severity of a particular medical condition based on parameter data for the measured physiological data parameters.

In the field of medicine physicians often desire to continuously monitor multiple physiological characteristics of their patients. Oftentimes, such monitoring of multiple physiological characteristics, or parameters, involves the use of several monitoring devices simultaneously, such as a pulse oximeter, a blood pressure monitor, a heart monitor, a temperature monitor, etc. These monitoring devices may be separate devices or elements within a larger multifunction patient monitoring device. Additional monitoring, treatment, and/or support devices and systems may further be connected to or associated with the patient, such as for delivering fluids, medication, anesthesia, respiration assistance, patient requested assistance, lab/imaging results, EMR/EHR notifications/alerts, etc. or analyzing various patient-related data to determine and alert a clinician to a condition or patient state (e.g., sepsis protocols, APACHE scores, early warning scores). Each of these devices and systems may generate one or more alarms to alert a clinician of a problem, which may be a problem with the patient's physiology or health status, or may be a technical problem with the monitoring and/or care delivery device. Thus, at any given time one or more devices may be generating alarms requiring the attention of a clinician.

### SUMMARY

This Summary is provided to introduce a selection of concepts that are further described below in the Detailed Description. This Summary is not intended to identify key or essential features of the claimed subject matter, nor is it intended to be used as an aid in limiting the scope of the claimed subject matter.

One embodiment of a computer-implemented method of monitoring a patient with respect to a particular medical condition includes receiving a long time interval of time-synchronized parameter data for each of at least two physiological parameters, and dividing the long time interval into multiple segments, wherein each segment contains a predefined time increment of the parameter data. A set of feature values are determined for the segment based on the parameter data in each segment, wherein the set of feature values includes a feature value for each of the predefined set of features related to the particular medical condition. With a trained machine learning model, assigning a weight to each of the predefined set of features, and then a risk severity index of the particular medical condition is calculated for the long time interval based on the set of feature values and weights.

In one embodiment, a patient monitoring system comprises one or more patient monitors measuring at least two physiological parameters from the patient and generating parameter data for each of the at least two measured physiological parameters and a processing system. The processing system is configured to receive a long time interval of time-synchronized parameter data for each of the at least two physiological parameters and divides the long time interval into multiple segments, each segment containing a predefined time increment of the parameter data. A set of feature values is determined for the long time interval based on the parameter data in each segment, wherein the set of feature values includes a feature value for each of a predefined set of features related to a particular medical condition. A trained machine learning model, such as a logistic regression model, assigns a weight to each of the feature values to calculate a risk severity index of the particular medical condition for the long time interval based on the set of feature values.

In another embodiment of a computer-implemented method of monitoring a patient with respect to a particular medical condition, a long time interval of time-synchronized parameter data is received, including parameter data for each of at least two physiological parameters. The long time interval of time-synchronized parameter data is divided into multiple segments, wherein each segment contains a predefined time increment of the parameter data. A slope of the parameter data is determined for each of the at least two physiological parameters in each segment. Each segment is then classified based on the slopes of the parameter data. A set of feature values is then determined for the segment based on the classification of each of the segments and the parameter data therein, wherein the set of feature values includes a feature value for each of a predefined set of features related to the particular medical condition. A visual code is then assigned to each segment based on the classification, and a progression map is generated for the long time series depicting the visual codes for each time segment. The progression map is then displayed on a display device.

Various other features, objects, and advantages of the invention will be made apparent from the following description taken together with the drawings.

### BRIEF DESCRIPTION OF THE DRAWINGS

The present disclosure is described with reference to the following Figures.

FIG. 1 is a graph depicting two physiological parameters, including SpO2 and respiratory rate, during the progression of acute respiratory distress syndrome.

FIG. 2 is a schematic diagram of an exemplary patient monitoring system according to one embodiment of the present disclosure.

FIGS. 3A-3C are exemplary progression maps depicting the progression and severity of an exemplary medical condition based on two monitored physiological parameters.

FIG. 4 is a flowchart demonstrating one embodiment of a method of implementing the trained machine learning module according to the present disclosure.

FIG. 5 is a flowchart demonstrating an exemplary method of training a machine learning model to receive a set of feature values and assign a weight to each of the feature values in order to generate a risk severity index for a particular medical condition.

FIG. 6 is a flowchart demonstrating one exemplary method of preprocessing raw parameter data according to the present disclosure.

FIGS. 7A-7D are graphs illustrating the preprocessing steps identified at FIG. 6.

FIG. 8 is a flowchart demonstrating steps for identifying an exemplary set of feature values according to one embodiment of the present disclosure.

### DETAILED DESCRIPTION

Current monitoring systems and methods effectively notify clinicians of the occurrence of an alarm event in monitoring data for a particular physiological parameter or within a monitoring modality. However, current patient monitoring systems and methods typically do not assess or provide information about comparative changes across different physiological parameters and/or monitoring modalities. Moreover, currently available patient monitoring methods and systems generally fail to analyze and/or account for long-term patterns or changes in physiological data, including patterns of smaller-magnitude changes occurring over the course of 12, 24, or 48 hours that do not, assessed individually, rise to the level of triggering an alarm.

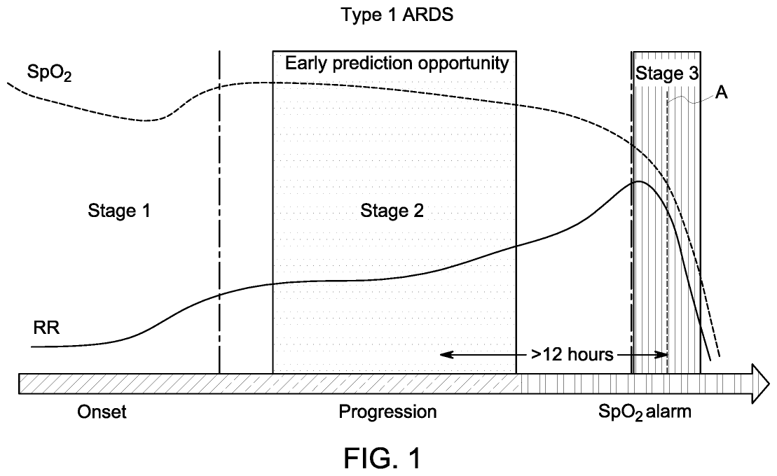

FIG. 1 depicts one example of a particular medical condition—namely, Type 1 acute respiratory distress syndrome (ARDS)—where early detection and intervention is extremely important for improving patient outcomes. The graph depicts peripheral oxygen saturation values (SpO2) and respiratory rate values (RR). In the depicted embodiment, the SpO2 and RR parameter data is characterizable into three stages relevant to the progression of Type 1 ARDS. In stage one ARDS, the SpO2 is declining and the RR is increasing. In stage two ARDS, the SpO2 is relatively stable and the RR is increasing. In stage three ARDS, the SpO2 and the RR are both decreasing rapidly. By the time the SpO2 alarm is generated (dashed line A), the patient condition is already rapidly declining and the patient condition is very severe.

The inventors have recognized that early prediction opportunity exists, such as illustrated in FIG. 1, where the progression of Type 1 ARDS can be detected prior to progression of the medical condition to a severe state. Given that ARDS, like many medical conditions, develops slowly over a relatively long period of time, the inventors have recognized that long-term assessment of patient monitoring data can be utilized to predict severity of certain medical conditions. In the depicted embodiment, the progression of SpO2 and RR occurs over a long time interval that is greater than 12-hours, and may even be greater than 24-hours. Accordingly, the inventors have recognized a need for systems and methods that monitor and compare parameter data from multiple parameters in order to detect patterns associated with particular medical conditions. Moreover, the inventors have recognized that machine learning models can be utilized to identify patterns of particular features in parameter data most associated with a particular medical condition. In one illustrative example discussed herein, SpO2 and RR are utilized over a 24-hour time interval to assess Type 1 ARDS.

In another example, medical conditions such as hypovolemic shock, internal bleeding, and/or aneurysm shock may be quantized by risk severity indexes based on physiological parameters including systolic blood pressure and heart rate. Hypovolemic shock occurs when intravascular volume decreases to the point of cardiovascular compromise, and may be due to severe dehydration through a variety of mechanisms or from blood loss. Bleeding may be either external or internal. Internal bleeding in some cases goes undetected in hypovolemic shock patients leading to health deterioration to critical condition and mortality. Severity of hypovolemic shock can be determined by a decrease in systolic blood pressure and a corresponding increase in heart rate over time. The inventors have recognized that this relation between HR and SysBP can be considered as Shock Stage clinical event and used to quantize severity by considering it as a feature for a machine learning model according to the disclosure provided herein.

The inventors have recognized that a significant number of medical conditions can be identified early and predicted based on comparative long-term analysis of patient monitoring data for two or more physiological parameters, and that such detections and predictions can occur prior to the onset of severe changes in the patient's physiological conditions. Such early detection is not available in current systems, which generally rely on triggering alarm conditions in order to analyze the patient's physiological condition and alert a clinician accordingly. However, such significant changes in the patient's physiological condition often indicate dire circumstances, and recognition of a medical condition at that point is often too late to provide preventive or early treatment as the medical condition is often already severe. Accordingly, the inventors have recognized that improved systems and methods are needed for analyzing patient monitoring data, including parameter data for multiple physiological parameters, in order to provide early detection of a patient's medical condition. As disclosed herein, the inventors have developed a prediction algorithm utilizing a machine learning model to calculate a risk severity index of a particular medical condition based on a long time interval of parameter data. The method involves analysis of parameter data for each of at least two different physiological parameters over a long time interval, such as 24-hours or more. In one embodiment, the method and system utilize a machine learning model, such as a logistic regression trained to assign a weight to each of a predefined set of features so as to calculate a risk severity index of a particular medical condition.

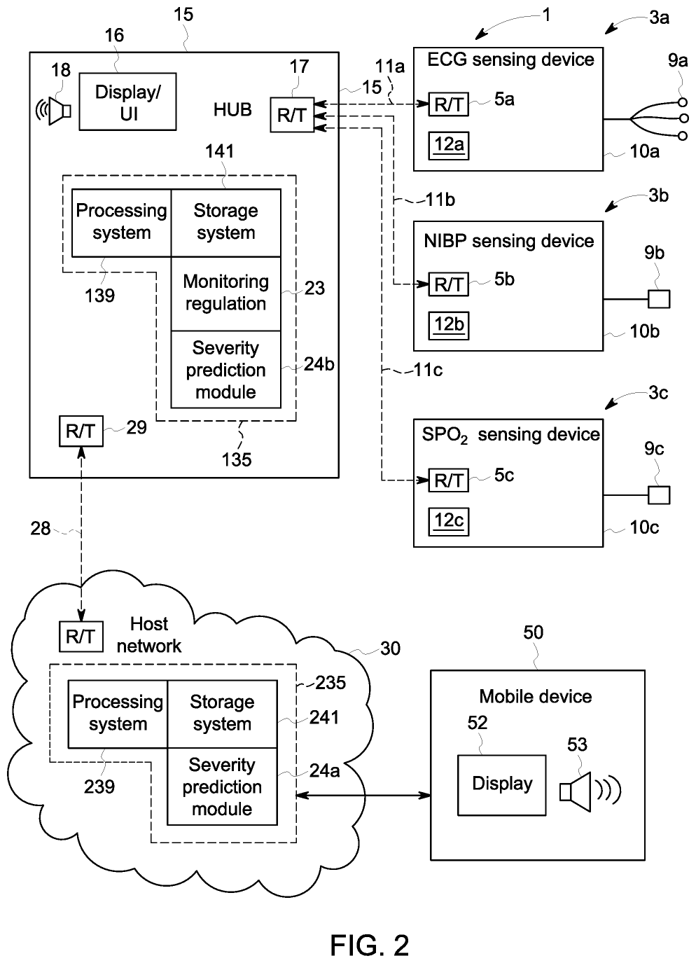

FIG. 2 depicts an exemplary embodiment of a patient monitoring system 1 including multiple sensing devices 3a-3c, each measuring a different physiological parameter from a patient. As will be known to a person having ordinary skill in the art, multi-parameter patient monitoring arrangements are common in the relevant field of patient monitoring, such as where multiple sensing devices (e.g. 3a-3c) communicate parameter data measured from the patient to a central device, or hub 15, or to a host network 30. As is also well-known, such communication of parameter data to the hub 15 or host network 30 may be by wired or wireless means. In various embodiments, the patient monitoring system 1 may monitor any set of two or more physiological parameters, and a wide variety of such multi-parameter monitoring arrangements are also well known.

In the example at FIG. 2, the patient monitoring system 1 includes three sensing devices 3a-3c in communication with hub 15. Each sensing device 3a-3c includes one or more sensors 9a-9c for measuring physiological parameters of a patient, and also includes a data acquisition device 10a-10c that receives the physiological parameter measurements recorded by the sensors 9a-9c and transmits a parameter dataset based on those measurements to the hub device 15 via communication link 11a-11c. In various embodiments, the communication link 11a-11c may be implemented via wired or wireless means, examples of which are well-known. The sensors 9a-9c may also be connected to the respective data acquisition device 10a-10c by wired or wireless means. The sensors 9a-9c may be any sensors, leads, or other devices available in the art for sensing or detecting physiological information from a patient, which may include but are not limited to electrodes, leadwires, or available physiological measurement devices such as pressure sensors, blood pressure cuffs, pulse oximetry sensors or the like.

In the depicted embodiment, a first sensing device 3a is an ECG sensing device having sensors 9a that are ECG electrodes. A second sensing device 3b is a non-invasive blood pressure (NIBP) sensing device with a sensor 9b that is a blood pressure cuff including pressure sensors incorporated therein. A third sensing device 3c is a peripheral oxygen saturation (SpO2) monitor having a sensor 9c that is a pulse oximetry sensor, such as a standard red-infrared pulse oximetry sensor configured for placement on a patient's fingertip. It should be understood that the patient monitoring system 1 of the present disclosure is not limited to the examples of sensing devices provided, but may be configured and employed to sense and monitor any physiological parameter of the patient. The examples provided herein are for the purposes of illustrating exemplary embodiments and should not be considered limiting.

The data acquisition device 10a-10c of each exemplary sensing devices 3a-3c may include an analog-to-digital (A/D) converter, which may be any device or logic set capable of digitizing analog physiological signals recorded by the associated sensor 9a-9c. For example, the A/D converter may be Analog Front End (AFE) devices. Each data acquisition device 10a-10c may further include a processing unit 12a-12c that receives the digital physiological data from the A/D converter and creates physiological parameter data for transmission to the hub device 15 and/or to the host network 30. Each data acquisition device 10a-10c may be configured differently depending on the type and function of sensing devices, and may be configured to perform various signal processing functions and/or sensor control functions. To provide just a few examples, the processing unit 12a in the ECG sensing device 3a may be configured to filter the digital signal from the ECG sensors 9a to remove artifact and/or to perform various calculations and determinations based on the recorded cardiac data, such as heart rate, QRS interval, ST segment/interval, or the like. The processing unit 12b in the NIBP monitor 3b may be configured, for example, to process the physiological data recorded by the sensors 9b in a blood pressure cuff to calculate systolic, diastolic, and mean blood pressure values for the patient. The processing unit 12c of the SpO2 sensing device 3c may be configured to determine a blood oxygenation value for the patient based on the digitized signal received from the pulse oximetry sensor 9c.

Accordingly, each processing unit 12a-12c may develop physiologic parameter data that, in addition to the recorded physiological data, also includes values measured and/or calculated from the recorded physiological data. The respective processing units 12a-12c may then control a receiver/transmitter 5a-5c in the relevant sensing devices 3a-3c to transmit the physiological parameter data to the hub device 15 via communication link 11a-11c. The physiological parameter data transmitted from the respective sensing devices 3a-3c may include the raw digitized physiological data, filtered digitized physiological data, and/or processed data indicating information about the respective physiological parameter measured from the patient. Additionally, one or more of the data acquisition devices 10a-10c may be configured to compare the physiological parameter data to one or more alarm thresholds to determine the presence of an alarm condition—i.e., detect an alarm event based on the physiological parameter data.

Upon detection of an alarm event by the respective sensing device 3a-3c, an alarm may be generated either by the sensing device 3a-3c (e.g., an auditory alarm via a speaker and/or visual alarm via a display) or the hub 15 (e.g., via speaker 18 and/or display 16), at a mobile device 50 (e.g., via speaker 53 and/or display 52), and/or a network access point (such as a central monitoring station or computer terminal at a nurse's station). Notice of the alarm may be transmitted from the respective sensing device 3a-3c to the hub 15, or may be detected at the hub 15 in the first instance as explained above. Further, the system may be configured in various ways for a clinician to silence the respective alarm, which may be provided via the respective sensing device 3a-3c, at the hub 15, or at some other location, such as via the mobile device 50.

The sensing devices 3a-3c may be networked to a central hub 15 (which could alternatively be a primary sensing device or other central device) that analyzes the parameter data and regulates the various sensing devices 3a-3c in the network. In certain embodiments, the hub 15 may communicate with a host network 30, such as a central network for a medical care facility. The sensing devices 3a-3c may communicate the parameter data to the host network 30, such as indirectly through the hub 15. For example, the hub may serve as an amplifier and/or router for communication between the sensing devices 3a-3c and the host network 30. In other embodiments (which may or may not include a hub 15), the sensing devices 3a-3c may communicate directly with the host network 30, such as by transmitting the parameter data recorded by the respective sensing devices directly to the host network 30 via a wireless network protocol and infrastructure. In the various embodiments, each sensing device 3a-3c may process its own physiological parameter data and determine its own alarming conditions, or such functions may be performed at the level of the host network 30.

It will be understood by a person having ordinary skill in the relevant art in light of this disclosure that the disclosed patient monitoring methods and systems may be executed by computing systems incorporated in the hub 15 (i.e., at the bedside patient monitoring system) or may be executed by a computing system incorporated in the host network 30. In the depicted example, the hub device 15 includes a computing system 135 having a processing system 139 and a storage system 141. The hub device 15 may serve to control the sensing devices 3a-3c, and thus may transmit operation commands to the respective sensing devices 3a-3c via the communication link 11a-11c to control their monitoring operations. The hub 15 may contain a monitoring regulation module 23 that is a set of software instructions stored in memory of the storage system 141 and executable by the processing system 139 to assess the physiological parameter data collected by the sensing devices 3a-3c, such as to detect an alarm event and to control the respective sensing devices 3a-3c according to the monitoring needs. For example, an alarm event may be determined by comparing the physiological parameter data collected by the one or more sensing devices 3a-3c with respective alarm limits to determine whether an alarm should be generated to alert the clinician of the patient's condition.

Likewise, the computing system 235 of the host network 30 comprises a processing system 239 communicatively connected to a storage system 241 so as to load and execute computer-readable instructions. While the description provided herein refers to a computing system 135, 235 and a processing system 139, 239, it is to be recognized that implementations of such systems can be performed using one or more processors, which may be communicatively connected, and such implementations are considered to be within the scope of the description. Each processing system 139, 239 can be implemented within a single processing device but can also be distributed across multiple processing devices or sub-systems that cooperate in executing program instructions. Each storage system 141, 241, which each store software that may include the severity prediction module 24, can comprise any storage media, or group of storage media, readable by processing system 139, 239, and capable of storing software. The storage system 141, 241 can include volatile and non-volatile, removable and non-removable media implemented in any method or technology for storage of information, such as computer-readable instructions, data structures, program modules, or other data. Each storage system 141, 241 can be implemented as a single storage device but may also be implemented across multiple storage devices or sub-systems Likewise, the storage media may be housed locally with the processing system 139, 239, or may be distributed in one or more servers, which may be at multiple locations and networked, such as in cloud computing applications and systems.

The system 1 includes a severity prediction module 24 which is a set of computer-readable instructions stored one or more storage systems 141, 241 and executable as described herein to calculate a risk severity index of a particular medical condition based on a long time interval of parameter data for two or more physiological parameters. The severity prediction module 24 may be executed on any computing system within the patient monitoring system 1, such as installed and executed on computing system 135 of the hub 15 or on computing system 235 of the host network 30. In still other embodiments, the severity prediction module 24 may be installed and executable on the mobile device 50, such as a smartphone or other mobile computing device operated by a clinician. In certain embodiments, the severity prediction module 24 may be installed on and executable by multiple computing systems within the overall patient monitoring system 1, or portions of the severity prediction module may be divided across the various computing systems (e.g., 135 and 235) within the overall system 1. In the depicted embodiment, the severity prediction module 24 is provided at both the computing system 235 of the host network (i.e., 24a) and on the computing system 135 of the hub 15 (i.e., 24b). Each severity prediction module 24a, 24b may perform the entirety of the functionality described herein, or each module may perform a portion of the functionality described herein.

In various embodiments, the computer-implemented method and system of monitoring a patient with respect to a particular medical condition includes receiving a long time interval of time synchronized parameter data for each of at least two physiological parameters, and processing the long time interval of parameter data using a trained machine learning model to identify patterns learned by the model and calculate a risk severity index of the particular medical condition based on a set of feature values identified in the parameter data. The set of features is defined based on the particular medical condition, as will be described in more detail below. In one embodiment, the long time interval of parameter data is divided into multiple segments, with each segment containing a predefined time increment of the parameter data. To provide one example, 24 hours of time-synchronized parameter data for two or more physiological parameters may be divided into 1 hour time segments. In another example, 12 hours of parameter data may be divided into 30-minute segments. In still another embodiment, five hours of time-synchronized parameter data for the two or more physiological parameters may be divided into 30-minute time segments, which may be particularly useful where hypovolemic shock is the medical condition being monitored.

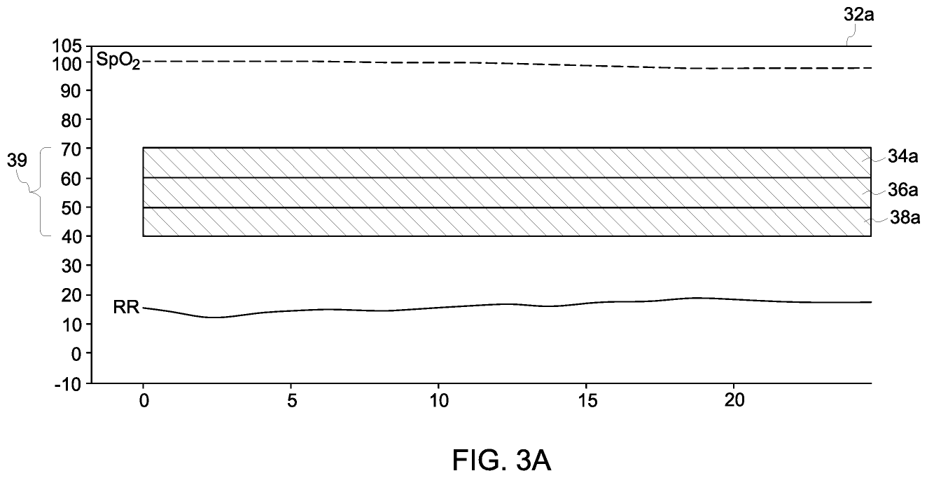

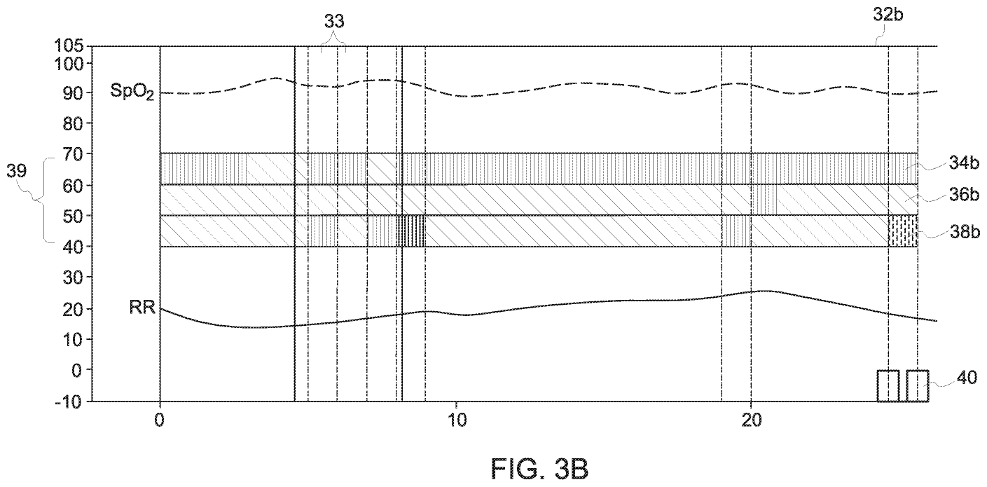

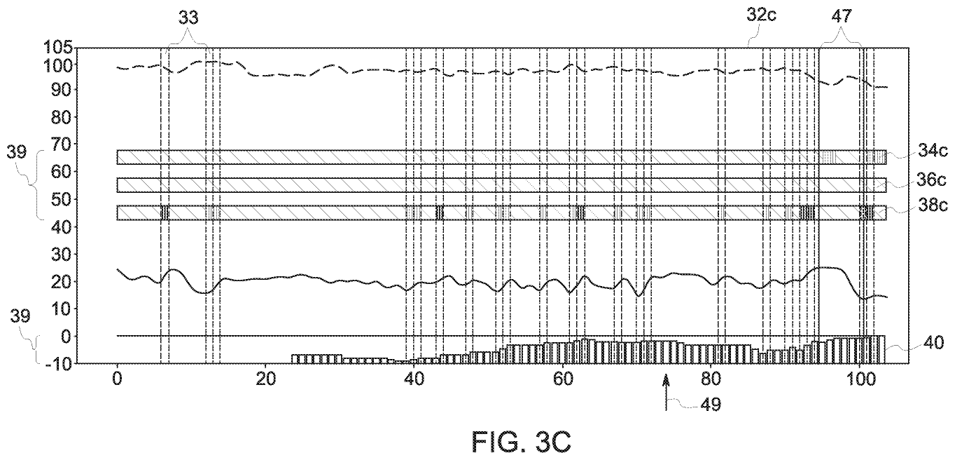

FIGS. 3A-3C provide visual depictions of exemplary long time intervals of time synchronized parameter data divided into segments of a predefined increment and analyzed accordingly. FIGS. 3A and 3B provide windows 32a, 32b showing a 24-hour time interval of time-synchronized parameter data for SpO2 and RR, as well as a progression map indicating feature values and risk severity for a particular medical condition (in this case, Type 1 ARDS). Window 32a exemplifies a 24-hour time interval where the patient is not exhibiting any indication of Type 1 ARDS, and window 32b depicts a 24-hour time interval of parameter data where Type 1 ARDS is exhibited.

The 24 hours of time synchronized parameter data (here, the SpO2 and RR data) are divided into segments 33 of one hour and the data is analyzed accordingly. Each segment of data is assessed individually, such as based on whether the parameter data in the segment exceeds an alarm threshold or a lesser threshold. Further, the slope of each parameter dataset in each segment 33 may be analyzed. For example, the slope may be classified into one of the above-described three stages of ARDS exemplified at FIG. 1—stage one where SpO2 is declining and RR is increasing, stage 2 where SpO2 is relatively stable and RR is increasing, and stage 3 where SpO2 and RR are both significantly declining. Each segment, and the 24-hour long interval as a whole, is assessed in terms of the predefined set of features based on the amplitude or slope of the parameter data.

Each segment may then be assigned a visual code based on the parameter data therein, such as based on the amplitude or slope of the data or based on the classifier. In the depicted examples, the visual code is a color code, where each segment 33 is assigned a color code according to one or more of the feature values. More particularly, in the depicted example each parameter data and the segment classifications are summarized by a color-coded progression map 39, including the SpO2 trend bar 34, the RR trend bar 36, and the classifier trend bar 38. Each trend bar is divided into segments 33 which are assigned a color code according to the relevant values therein. Where the parameter data is stable, exemplified in window 32a, each segment is assigned a color code (e.g., green) representing that the parameter data is normal and does not meet any threshold amplitude or slope or meet the criteria for ARDS stage classifications. For example, where the slopes of the SpO2 and RR data meet the requirements of stage one, then the relative segment 33 of the classifier trend bar 38 is assigned a first color (e.g., pink). Where the parameter data in the segment meets the requirements of stage 2 for the ARDS assessment, the relevant segment 33 of the trend bar 38 is colored a second color (e.g., orange). If the parameter data in the segment meets the requirements of stage 3 of the ARDS assessment, then the relevant portion of the trend bar corresponding to that segment 33 is assigned a third color (e.g., red or violet). Similarly, the trend bars 34, 36 for each parameter may be color-coded according to the data values in each segment for the respective physiological parameter. For example, the SpO2 trend bar 34 may be color-coded according to the magnitude or slope of the values therein, such as the magnitude of the SpO2 with respect to one or more low SpO2 thresholds. Likewise, the RR trend bar 36 may be color-coded according to the magnitude or slope of the parameter data in the relevant segment 33, such as the RR data values with respect to one or more high respiration thresholds. The low SpO2 thresholds and the high RR thresholds may include alarm thresholds as extreme thresholds, and may also include additional moderate thresholds, where a color code is associated with each threshold and assigned accordingly.

In window 32a where ARDS is not detected or present, the trend bars 34a, 36a, 38a are consistently green across the entire 24-hour time interval. By contrast, in window 32b where the risk severity index for ARDS is high, the trend bars 34b, 36b, 38b show multiple colored segments associated with higher risk. However, as can be seen from the example in FIG. 3C, the risk severity index can detect certain patterns in the parameter data that are not otherwise visible by viewing the parameter data, alone. The SpO2 trend bar 34c and the RR trend bar 36c of the progression map 39 in FIG. 3C generally show that the SpO2 and RR parameter values, assessed individually, do not cross the relevant threshold values indicating a problem. However, the classifier trend bar 38, which is based on a comparison of the SpO2 slope and the RR slope, identifies multiple problematic segments 33 based on the comparative slope values characterizing stages one, two and three for ARDS. Accordingly, this long-term assessment method of comparing these parameter data over long time intervals can provide information that examining any single parameter, alone, cannot provide. Likewise, examining any short period of parameter data also cannot provide the type of information needed for early detection of certain medical conditions such as ARDS.

At the bottom of each time window 32a-32c is an exemplary visual indicator 40 of the risk severity index. As described herein, a risk severity index of the particular medical condition may be calculated based on the set of feature values for the long time interval of time-synchronized parameter data. In one example, the risk severity index is a probability of the particular medical condition calculated based on the set of feature values. For instance, the risk severity value may be a value between 0 and 1 that indicates a corresponding probability (0%-100%) of the medical condition, where higher probability values closer to 1 indicate a higher risk severity of the particular medical condition and probability values closer to 0 indicate a lower severity risk.

The risk severity index is calculated based on the long time interval, such as 24 hours of parameter data as shown in the depicted embodiments. In FIG. 3B, the risk severity index is calculated for the depicted 24-hour interval of SpO2 and RR parameter data and is indicated by the risk severity visual indicator 40. The risk severity visual indicator is aligned with the last segment of the 24-hour long time interval, as it is calculated based on the entire time interval data.

The risk severity index is a function of a predefined set of feature values that are clinically relevant features weighted using a machine learning model, which is described in more detail below. The window 32c at FIG. 3C depicts multiple risk severity indexes, which are calculated for sliding 24-hour time intervals for over 100 hours of parameter data. The progression map 39 shows the risk severity visual indicators 40 starting after 24 hours of parameter data and updated each hour thereafter. This particular example illustrates a dataset for a patient who developed severe Type 1 ARDS that went undetected until SpO2 alarms were generated (represented at lines 47), at which point treatment was too late and a critical medical event (which in this case was terminal for the patient) could not be prevented.

However, earlier detection of the ARDS condition could have occurred using the methods and systems disclosed herein. Upon earlier detection, medical intervention could have been administered and likely prevented the onset of severe ARDS. Namely, assessment of the risk severity index, such as assessment over a predefined amount of time, can yield an early indication of medical conditions, such as ARDS. For example, an alarm may be generated if at least a threshold number of the most recent risk severity index values are greater than a threshold. For instance, if the risk severity index is at or above 80% for four consecutive hours then an alarm may be generated indicating detection of the particular medical condition and the need for medical intervention. In the depicted example, the arrow 49 indicates alarm generation where the risk severity index is greater than or equal to 80% for four consecutive hours. This is just one example, and other thresholds and time periods may be more clinically relevant. Alternatively or additionally, alarms may be generated if the risk severity index exceeds a high threshold for even one-time interval calculation, such as exceeding a high threshold of 90%.

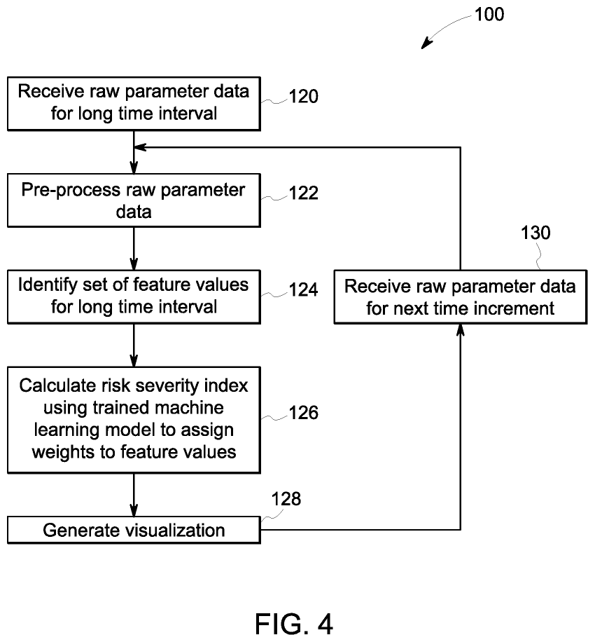

FIG. 4 depicts one embodiment of a computer-implemented method 100 of monitoring a patient with respect to a particular medical condition. For instance, the flowchart represents steps executed by the severity prediction module 24 on one or more computing systems (e.g. computing system 135 and the hub 15 or computing system 235 and the host network 30). Raw parameter data is received at step 120 for the long time interval. As described above, the long time interval may be any period sufficient to detect the particular medical condition. For example, the long time interval for Type 1 ARDS may be any value between 30 minutes and 48 hours. For many medical conditions, the long time interval will be at least six hours, and often 12 hours or 24 hours.

Once the first long time interval of raw parameter data for at least two parameters is received at step 120, the raw parameter data is pre-processed at step 122 in order to prepare the data for feature value assessment. A set of feature values for the long time interval are identified at step 124. The feature values are defined based on identified clinical patterns of the particular medical condition, such as based on amplitude or slope assessments of the parameter data. Exemplary sets of feature values for exemplary medical conditions are described in more detail below. The risk severity index is then calculated at step 126 using a trained machine learning model to assign weights to the feature values. A visualization is generated at step 128, such as exemplified in FIGS. 3A-3C. The severity prediction module 24 then waits for the next time increment of raw parameter data, which is received at step 130. The predefined time increment defines the segments of parameter data, which must be less than the long time interval value. Generally, the predefined time increment is between one minute and one hour, but may be longer than 1 hour. In the examples depicted at FIGS. 3A-3C, the predefined time increment is one hour and the long time interval is 24 hours. However, other time intervals and time increments may be used depending on the amount of time needed to detect patterns relevant to the particular medical condition being assessed.

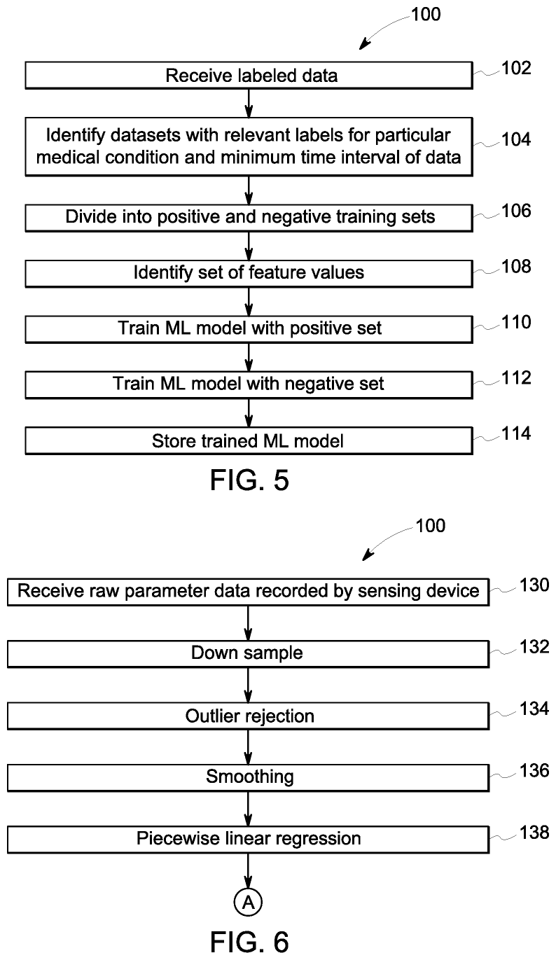

FIG. 5 depicts exemplary steps for training the machine learning model to receive a set of feature values and assign a weight to each of the feature values therein so as to calculate a risk severity index of a particular medical condition. As will be understood by a person of ordinary skill in the relevant art, different types of machine learning models may be utilized. In one example, the machine learning model is a logistic regression model trained based on a dataset comprising labeled long time intervals of parameter data. The labeled long time intervals are labeled as either positive for the particular medical condition or negative for the particular medical condition. The logistic regression model has the benefit of being a simple and transparent model, providing observing clinicians a logical understanding of the calculated risk severity index because the weights given to each of the features (clinical events) can be determined. However, other machine learning models may be utilized to calculate the risk severity index, such as a support vector machine (SVM), a multilayer perceptron (MLP), a convolutional neural network (CNN), linear discriminant analysis (LDA), baggage and random forest ensemble algorithms, or naïve Bayes classifiers. Labeled data is received at step 102, and the data is sorted at step 104 to identify relevant training data, such as select parameter data recordings of a length equal to the long time interval labeled as positive or negative for the particular medical condition. Once the training dataset is identified, it is divided into positive and negative training sets at step 106—namely, based on the positive and negative labels for the particular medical condition. The predefined set of feature values are identified at step 108 within each long time interval of each patient dataset. The set of feature values and positive/negative labels are then used to train the machine learning model. The machine learning model is trained with the positive dataset at step 110 and with the negative dataset at step 112, and the resulting trained machine learning model is able to assign a weight to each of the set of feature values in order to calculate the risk severity index for the particular medical condition. The trained machine learning model is stored at step 114. The stored machine learning model is then utilized to calculate the risk severity index. Using a trained logistic regression model, for example, the severity index is represented as a function of the set of feature values as follows:

$$h ( x ) = \frac{1}{1 + {e}^{- {θ}^{T} f}} = P ( Y = RD \vert f ; θ )$$

where h(x) is the severity index (the probability of the particular medical condition), f is the predefined set of features, and θ^{T} is the coefficients returned by the best trained logistic regression model.

In development of one embodiment, the inventors utilized datasets from the MIMIC dataset, which is an openly-available dataset of more than 40,000 critical care patients developed by the MIT Lab for Computational Physiology. This was the labeled data (step 102) used to train the logistic regression model. From the labeled data, datasets were identified (step 104) with relevant labels for a particular medical condition, which in this embodiment were positive and negative labels for Type 1 ARDS. Furthermore, the datasets were also assessed based on the time interval of data, where the training datasets contained at least the long time interval of data so that the risk severity index can be determined appropriately. In various embodiments, other features of the datasets may also be required for identifying the training dataset. For instance, in training the logistic regression model for ARDS detection, it may be preferable to limit the training data to parameter datasets where the patient was not on a ventilator.

FIG. 6 represents a set of method steps for pre-processing the raw parameter data, both for training purposes and for implementation of the disclosed risk severity calculation. The raw parameter data recorded by the sensing device is received at step 130. The parameter data may be downsampled at step 132, or other numerics may be applied to generate a representation of the waveform data a very low sample rate, such as one sample/min. Given the analysis of the long term trend, the lower sample rate may be more suitable for the risk severity index calculation. However, in other embodiments, the full waveform data (such as at 240 samples per minute) may be used.

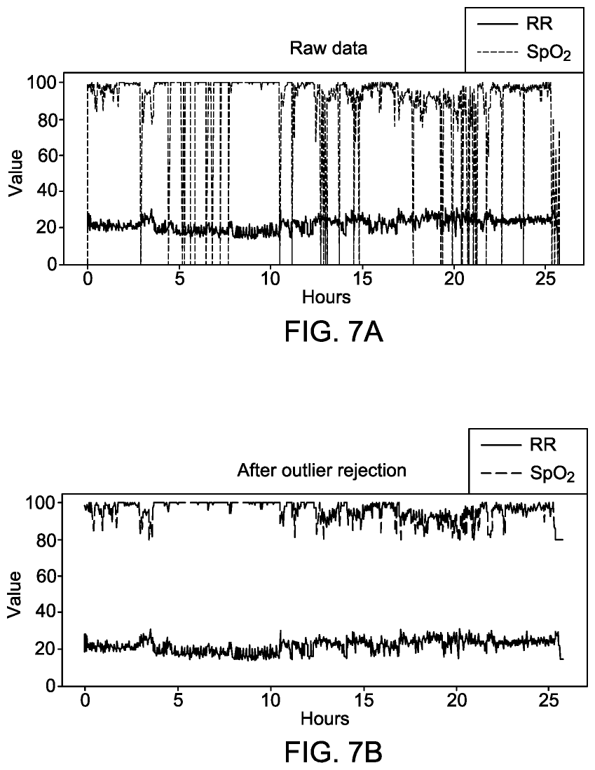

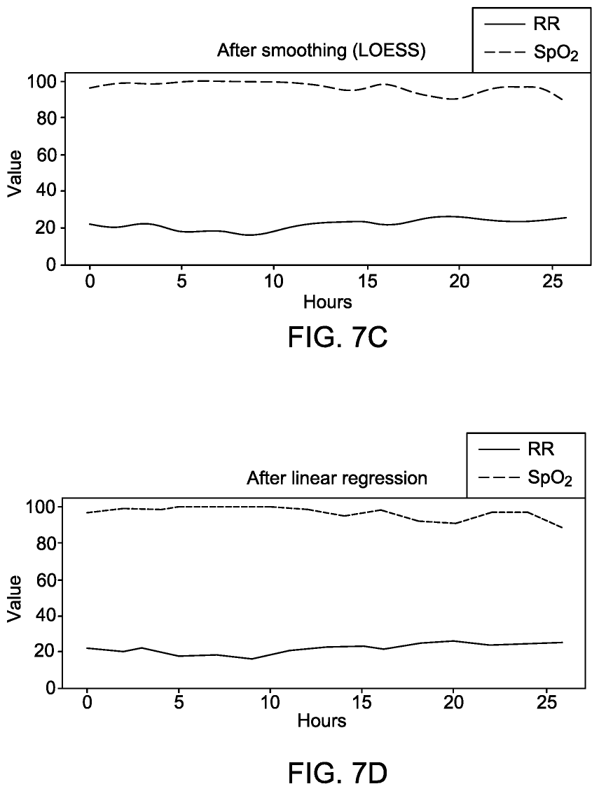

The downsampled raw parameter data is then filtered using outlier rejection at step 134 to reduce noise present in the parameter data. For example, Chauvenet's rejection criteria for outlier rejection may be utilized, followed by interpolation using backfilling to resample to the down-sampled frequency (e.g. one sample/min). For example, outlier rejection by Chauvenet's criterion may utilize a maximum allowable deviation of 2.5. Comparison of FIGS. 7A and 7B demonstrate the benefit of outlier rejection, where the raw parameter data in FIG. 1A is noisy, such as due to patient movement, optical interference, problems with sensor placement, etc. FIG. 7B shows the same parameter data after outlier rejection, which demonstrates that much of the artifact is eliminated.

Returning to FIG. 6, the parameter data is smoothed at step 136 using a smoothing algorithm. For example, the parameter data may be smoothed using locally estimated scatterplot smoothing (LOESS) or locally weighted scatterplot smoothing (LOWESS) local polynomial regression. Thereby, a piece-wise quadratic polynomial is fitted to the data values to smooth the data. This method has a distinct advantage over the general framework of least squares regression, which generalizes the smoothing of the entire time series with a single function. Relevant to the ARDS detection embodiment using a 24-hour time interval and 1-hour segments sampled at one sample/min, a window size of three hours may be used for the smoothing. In this example, since the sampling rate is one sample/min, a single piece of quadratic polynomial covers 180 sample points. FIG. 7C depicts the parameter data after smoothing using LOESS with the 3-hour window size.

Referring back to FIG. 6, the denoised and smoothed parameter data is then fitted with piece-wise linear regression (PLR). PLR helps model the time series into separate segments, thereby providing linear approximation for each segment. In the depicted examples, 1-hour segments are defined for linear regression fitting, as this helps to capture the long-term trend in the long time interval (e.g., 24 hours). The PLR fits a line to the parameter data in each 1-hour segment. Assuming the one sample/min sample rate, each 1-hour segment fits a line to 60 sample points. Accordingly, the slope of the segment can be quantified for steepness and utilized for the feature value detection, such as classified into various stages defined for a particular medical condition. FIG. 7D is a graph representing the exemplary parameter data after PLR.

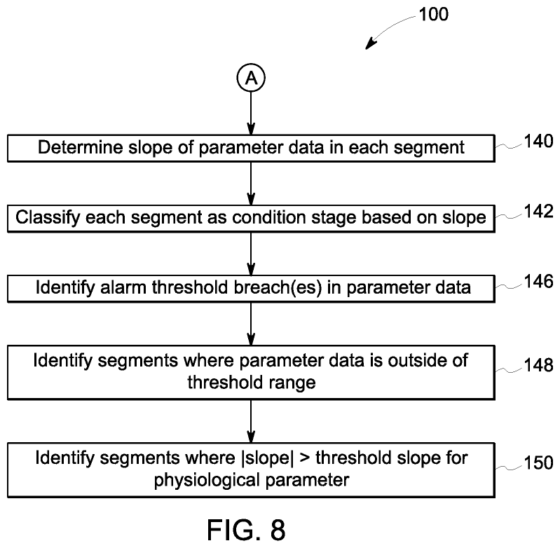

After the parameter data has been pre-processed it can be analyzed with respect to a predefined set of features in order to generate the set of feature values that will be analyzed by the trained machine learning model. FIG. 8 represents steps for analyzing parameter data with respect to a predefined set of features determined to be clinically relevant for detecting ARDS. The exemplary predefined set of features for ARDS detection include the following:
Whether RR Breach occurred in the long time interval
Whether SpO2 Breach occurred in the long time interval
Number of segments where RR is above RR threshold(s)
Number of segments where SpO2 is below SpO2 threshold(s)
Number of occurrences of Stage 1 ARDS
Number of occurrences of Stage 2 ARDS
Number of occurrences of Stage 3 ARDS

In order to generate the set of feature values for the above-listed predefined set of features, the step represented at FIG. 8 may be executed. A slope of the parameter data in each segment is determined at step 140, which follows from the piece-wise linear regression described above. Each segment is then classified at step 142 according to the stage definitions described above. The number of segments where each of stage one, two, and three ARDS is present can then be counted. At step 144, the parameter data for the long time interval is assessed to identify whether alarm threshold breaches are present. For instance, an RR breach may be identified where the respiration rate exceeds 25 cycles/min, and a SpO2 breach may be identified where the SpO2 parameter data is less than 90. Steps are also executed to identify the number of segments where the parameter data is outside of a threshold range (which includes one or more thresholds that are different, and less extreme, than the alarm threshold values). As described above with respect to FIGS. 3A-3C, multiple threshold values may be set for assessing the amplitude of the parameter data at step 146. In various embodiments, additional features may be identified other than those listed above. An additional example is represented at step 148 where a magnitude of the slope in each parameter data segment is compared to a threshold slope for the relevant physiological parameter. Similar to the amplitude thresholds, multiple slope thresholds may be provided to yield a detailed categorization and progression tracking of the change in the relevant physiological parameter.

In an example where the risk severity index is calculated for monitoring hypovolemic shock, a trend of physiological parameters, including heart rate and systolic blood pressure, can be monitored over time and used to quantize severity of shock. Also, in addition, a high respiratory rate (e.g. greater than 20 breaths/min in adult population), severe hypotension (systolic blood pressure<90 mmHg) and pronounced tachycardia (heart rate>120 bpm) can help quantize severity. In this clinical scenario, a 5-hour long time interval and 30-minute segments sampled at one sample/min, and a window size one and a half hours (3*30 minutes) may be used for the smoothing. In this example, since the sampling rate is one sample/min, a single piece of quadratic polynomial covers 90 sample points. After the parameter data has been pre-processed, it can be analyzed with respect to a predefined set of features in order to generate the set of feature values that will be analyzed by the trained machine learning model. For example, the set of features in this clinical scenario may include:
Number of segments where RR is above RR threshold(s)
Number of segments where HR is above HR threshold(s)
Number of segments where SysBP is above SysBP threshold(s)
Whether RR Breach occurred in the long time interval
Whether HR Breach occurred in the long time interval
Whether SysBP Breach occurred in the long time interval
Number of occurrences of Shock Stage Clinical event for hypovolemic shock

This written description uses examples to disclose the invention, including the best mode, and also to enable any person skilled in the art to make and use the invention. Certain terms have been used for brevity, clarity, and understanding. No unnecessary limitations are to be inferred therefrom beyond the requirement of the prior art because such terms are used for descriptive purposes only and are intended to be broadly construed. The patentable scope of the invention is defined by the claims and may include other examples that occur to those skilled in the art. Such other examples are intended to be within the scope of the claims if they have features or structural elements that do not differ from the literal language of the claims, or if they include equivalent features or structural elements with insubstantial differences from the literal languages of the claims.

We claim:

1. A computer-implemented method of monitoring a patient with respect to a particular medical condition, the method comprising:
receiving a long time interval of time-synchronized parameter data for each of at least two physiological parameters;
dividing the long time interval into multiple segments, each segment containing a predefined time increment of the parameter data for each of the at least two physiological parameters;
determining a set of feature values for the long time interval based on the parameter data in each segment, wherein the set of feature values includes a feature value for each of a predefined set of features related to the particular medical condition;
with a trained machine learning model, assigning a weight to each of the predefined set of features; and
calculating a risk severity index of the particular medical condition for the long time interval based on the set of feature values and the weights.

2. The method of claim 1, wherein the trained machine learning model is a logistic regression model, and further comprising training the logistic regression model based on a dataset of labeled long time intervals of parameter data for each of the at least two parameters, wherein the labeled long time intervals are labeled as either positive or negative for the particular medical condition.

3. The method of claim 1, further comprising determining a slope of the parameter data in each segment, wherein the set of feature values is based further on the slopes in each segment.

4. The method of claim 3, further comprising classifying each segment based on the slopes of the parameter data for each of the at least two physiological parameters, wherein each set of feature values for the long time interval includes the classification for the segments.

5. The method of claim 4, further comprising:
assigning a color code to each segment based on the classification;
generating a progression map for the long time interval depicting the color codes for each time segment;
displaying the progression map on a display device and updating the progression map on the display device after each predefined time increment.

6. The method of claim 1, further comprising recalculating the risk severity index at an interval equal to the predefined time increment such that the long time interval represents a sliding interval of most recent time-synchronized parameter data for each of at least two physiological parameters.

7. The method of claim 6, further comprising generating an alarm if at least a threshold number of most recent risk severity indexes exceed a threshold risk value.

8. The method of claim 1, wherein the particular medical condition is acute respiratory distress syndrome (ARDS) and the at least two physiological parameters include SpO2 and respiration rate (RR).

9. The method of claim 8, wherein the predefined set of features includes at least three of:
a. whether an RR alarm threshold was breached during the long time interval,
b. whether a SpO2 alarm threshold was breached during the long time interval,
c. a number of segments in the long time interval where a respiration rate value exceeds an RR threshold,
d. a number of segments in the long time interval whether a SpO2 value is less than a SpO2 threshold,
e. a number of segments having a stage 1 ARDS classification type,
f. a number of segments having a stage 2 ARDS classification type, and
g. a number of segments having a stage 3 ARDS classification type.

10. The method of claim 1, further comprising, prior to dividing the long time interval into multiple segments, performing outlier rejection for each of the at least two parameters and smoothing the parameter data for each of the at least two parameters.

11. A patient monitoring system comprising:
one or more patient monitors measuring at least two physiological parameters from a patient and generating parameter data for each of the at least two measured physiological parameters;
a processing system configured to:
receive a long time interval of time-synchronized parameter data for each of the at least two physiological parameters;
divide the long time interval into multiple segments, each segment containing a predefined time increment of the parameter data for each of the at least two physiological parameters;
determine a set of feature values for the long time interval based on the parameter data in each segment, wherein the set of feature values includes a feature value for each of a predefined set of features related to a particular medical condition;
use a trained machine learning model to assign a weight to each feature value in the set of feature values, and
calculate a risk severity index of the particular medical condition for the long time interval.

12. The system of claim 11, wherein the processing system is further configured to determine a slope of the parameter data for each of the at least to parameters in each segment, and to determine set of feature values based further on the slopes for each segment.

13. The system of claim 12, wherein the processing system is further configured to classify each segment based on the slopes of the parameter data for each of the at least two physiological parameters, wherein each set of feature values for the long time interval includes the classification for the segments.

14. The system of claim 13, further comprising a display device, and wherein the processing system is further configured to:
assign a color code to each segment based on the classification;
generate a progression map for the long time interval depicting the color codes for each time segment; and
display the progression map on the display device.

15. The system of claim 11, wherein the trained machine learning model is a logistic regression model trained based on a dataset comprising labeled long time intervals of parameter data for each of the at least two parameters, wherein the labeled long time intervals are labeled as either positive or negative for the particular medical condition.

16. The system of claim 11, wherein the processing system is further configured to generate an alarm if at least a threshold number of most recent risk severity indexes exceed a threshold risk value.

17. The system of claim 11, wherein the long time interval is at least 24 hours and the predefined time increment is at least 1 hour.

18. The system of claim 17, wherein the particular medical condition is acute respiratory distress syndrome (ARDS) and the at least two physiological parameters include SpO2 and respiration rate; and
wherein the predefined set of features includes:
a. whether an RR alarm threshold was breached during the long time interval,
b. whether a SpO2 alarm threshold was breached during the long time interval,
c. a number of segments in the long time interval where a respiration rate value exceeds an RR threshold,
d. a number of segments in the long time interval whether a SpO2 value is less than a SpO2 threshold,
e. a number of segments having a stage 1 classification type,
f. a number of segments having a stage 2 classification type, and
g. a number of segments having a stage 3 classification type.

19. A computer-implemented method of monitoring a patient with respect to a particular medical condition, the method comprising:
receiving a long time interval of time-synchronized parameter data for each of at least two physiological parameters;
dividing the long time interval into multiple segments, each segment containing a predefined time increment of the parameter data for each of the at least two physiological parameters;
determining a slope of the parameter data for each of the at least two physiological parameters in each segment;
classifying each segment based on the slopes of the parameter data;
determining a set of feature values for the long time interval based on the classifications of each of the segments and the parameter data in each segment, wherein the set of feature values includes a feature value for each of a predefined set of features related to the particular medical condition;
assigning a visual code to each segment based on the classification; and
generating a progression map for the long time interval depicting the visual codes for each time segment, and then displaying the progression map on a display device.

20. The method of claim 19, further comprising:
calculating a risk severity index of the particular medical condition for the long time interval based on the set of feature values;
assigning a visual indicator based on the risk severity index;
displaying the risk severity visual indicator on the display such that it is visually aligned with the visual code for a most recent segment in the long time interval.
